(19) United States
(12) Patent Application Publication
(10) Pub. No.: US 2025/0146031 A1
(43) Pub. Date: May 8, 2025

(54) INSECT-DERIVED, ENZYME-MODIFIED LIPID COMPOSITION

(71) Applicant: PROTIX B.V., s-Hertogenbosch (NL)

(72) Inventors: Aman PAUL, s-Hertogenbosch (NL); Monika DALIM, s-Hertogenbosch (NL)

(73) Assignee: PROTIX B.V., s-Hertogenbosch (NL)

(21) Appl. No.: 18/838,306

(22) PCT Filed: Feb. 16, 2023
(86) PCT No.: PCT/NL2023/050075

§ 371 Date: Aug. 14, 2024

(30) Foreign Application Priority Data
Feb. 22, 2022 (NL) 2031036

(51) Int. Cl. C12P 7/6418; A61K 31/232; A61K 35/64; A61P 31/04; C11C 1/04; C11C 3/10; C12N 9/20

(52) U.S. Cl. CPC C12P 7/6418; A61K 31/232; A61K 35/64; A61P 31/04; C11C 1/045; C11C 3/10; C12N 9/20; C12Y 301/01

(57) ABSTRACT

The present invention relates to lipid compositions derived from insects. The intrinsic chemical/physical properties of insect derived lipids limit the possibilities for widespread and large-scale use. An enzymatic process for the partial hydrolysis and transesterification of insect extracted lipids is provided. The process alters the ratio of triglyceride (TAG), diglycerides (DAG), monoglycerides (MAG) and free fatty acids (FFA) of the crude insect lipid and also results in the transesterification of fatty acids on glyceride backbone yielding a lipid composition that combines an increased dispersibility in water, a more favourable hardness profile (as a function of temperature) and a melting point slightly above ambient temperature. The compositions of the invention further have good nutritive properties as well as some interesting functional properties, e.g. anti-microbial effects. The invention relates to these enzyme-modified lipid compositions, to the methods for producing them, as well as to their uses as an ingredient in an alimentary product.

### FIELD OF THE INVENTION

The present invention relates to lipid compositions derived from insects, in particular lipid compositions derived from insect larvae, more in particular lipid compositions derived from larvae of the species Black Soldier Fly, that have been treated with an enzyme, in particular an enzyme that has hydrolysis and (trans) esterification activity. The invention also relates to a method for producing these enzyme-modified lipid compositions from insects, in particular from insect larvae, more in particular from the larvae of Black Soldier Flies, and to the lipid compositions that are obtainable by such methods. The invention further relates to uses of the enzyme-modified insect lipid compositions of the invention and to products comprising an enzyme-modified insect lipid composition of the invention as an ingredient.

### BACKGROUND OF THE INVENTION

In the past decades, there has been a growing interest in insects as a food source, especially in view of the growth of global population and malnutrition in the developing world. Insects are rich in proteins and sometimes fats and represent a relatively high caloric value. Insects are associated with high feed conversion efficiency and lower emissions of greenhouse gases than conventional livestock. Insect rearing processes have been developed and implemented that can produce and process large numbers/volumes of insects, often in a highly automated fashion. Insects that are (nowadays) typically ‘farmed’ include flies, bugs, mosquitos, butterflies, moths, cicadas, termites, bees, ants, wasps, beetles, grasshoppers, or crickets. Insect farming essentially entails breeding and rearing of insects in a manner tailored to maximize conversion of the substrate into insect and larval biomass. In general, most of the attention focuses on protein yield, in view of the high global demand for protein and the large environmental impact associated with protein production in livestock.

Exemplary methods for processing of insects to separate protein from other fractions are described in published patent applications WO2014/123420 and WO 2021/125956.

As can be inferred from WO2014/123420 and WO 2021/125956 documents, lipids make up a significant part of insect bodies and are a major co-product of protein production with insects.

Lipids constitute an important raw material for food and feed industries, where they can serve a large variety of purposes, ranging simply from a dietary energy source to structuring and texturizing of food products, all dependent on their specific chemical, physical and nutritional properties. Each oil or fat contains a wide variety of different lipid structures, defined by the fatty acid content/makeup and the regiochemical distribution of fatty acids on the glycerol backbone. The value of lipids increases greatly as a function of fatty acid composition as well as purity. High purity can be achieved by processing such as fractional chromatography or distillation, which is typically very costly, while yields are often limited.

Traditionally, the food and feed industry primarily relied on fish and rendered animal fats. In modern days, there is a trend to substitute such animal derived lipids with vegetable lipids, such as lipids obtained from the processing of oilseeds from plants like rice bran oil, rapeseed (canola), sunflower, olive, palm, coconut or soy. This increasing demand for vegetable oil, unfortunately, has become one of the main drivers of deforestation globally and the growing of vegetable oil crop also tends to compete for systemic resources with food availability.

The lipid fractions obtained as a co-product from insect (protein) production, may constitute an important, sustainable alternative to certain vegetable oils, such as palm kernel oil and coconut fat. To date, however, uses of insect derived lipid compositions are still very limited; they are marketed/used, on a modest scale, for feeding to livestock and pets.

Ewald et al. (Fatty acid composition of black soldier fly larvae (Hermetia illucens)—Possibilities and limitations for modification through diet. Waste Management 102 (2020) 40-47) investigated whether the fatty acid profile of insect lipid fractions could be enhanced, through modification of the rearing substrate, with a specific focus on increasing PUFA content. Based on their findings, Ewald et al. conclude that the possibilities for modification of the fatty acid profile appear limited and that it would be advisable to focus on the major fatty acid constituent, i.e. the SFA, in particular lauric acid, rather than the minor constituents (such as EPA and DHA) when evaluating possibilities for future applications of the BSFL fat.

Caligiani et al. (Influence of the killing method of the black soldier fly on its lipid composition. Food Research International 116 (2019) 276-282) discuss some of the intricacies of insect fat extraction and processing. Caligiani et al. note that, at the time (i.e. in 2019), BSF fat was still mainly used for the production of biodiesel and that much more investigation was needed to support exploitation of higher added value uses in the food/feed sector. For that reason Caligiani et al. studied the composition of BSF lipids and the effect of different killing and storage on their quality. Global fatty acid and sterol profiles, determined by GC-MS, were only slightly affected by the killing procedure, while lipid classes distribution, determined by ¹H NMR, strongly changed. Prepupae killed by freezing showed a drastic reduction of acylglycerols during storage and a relevant release of free fatty acids, likely due to activation of lipases. Prepupae killed by blanching have a stable lipid fraction constituted mainly by triacylglycerols. Caligiani et al. conclude that, for eventual food applications of BSF oils, a thermal pre-treatment on living larvae/prepupae is necessary to inactivate lipase and preserve an intact/stable lipid fraction.

Despite the fact that insect lipids have favourable nutritive and even certain beneficial ‘functional’ properties, and despite the fact that, from the environmental standpoint, they constitute a very attractive substitute for traditional animal and/or vegetable derived lipids, the use of insect lipids has not really caught on in the (global) food and/or feed markets yet. It is becoming increasingly clear that the intrinsic chemical and/or physical properties of insect lipids often hamper the (facile) replacement of traditional (animal and/or vegetable derived) lipids in many applications. Insect lipid fractions obtained with the known processing techniques, for instance, have proven to be disfavoured for certain applications because they become too hard at lower temperatures (around the freezing point). This is particularly relevant in case of solid fat feed or food compositions that are produced, stored and/or used at below-ambient temperature. It has also been reported that dispersibility of insect lipid fractions in water is too low and that (o/w) emulsion formulations produced with crude insect lipids are often very difficult to stabilize (even with emulsifying agents at acceptable amounts).

These and other intrinsic chemical/physical properties of insect derived lipids limit the possibilities for widespread and large-scale use, as a result of which environmental benefits and economic potential of insect farming still remain partly untapped. There is thus a need to provide an improved insect lipid based product that has more favorable chemical, physical, and/or nutritional properties, and, hence, is amenable for more widespread and versatile application in feed and food industries.

### SUMMARY OF THE INVENTION

To this end, the present inventors have developed an enzymatic process for the partial hydrolysis and transesterification of insect extracted lipids. The process alters the ratio of triglyceride (TAG), diglycerides (DAG), monoglycerides (MAG) and free fatty acids (FFA) of the crude insect lipid and also results in the transesterification of fatty acids on glyceride backbone. The enzymatic process of the invention, unexpectedly, yields a lipid composition that has, amongst other things, a reduced hardness at lower temperatures and a significantly improved water-dispersibility, without detriment to other chemical, physical and/or nutritional properties, such as the melting point of the lipid composition, as is illustrated in the experimental part of this document.

The present enzyme-modified insect derived lipid composition has been characterized by determining, amongst other things, the (relative) levels of triacylglycerol, diacylglycerol, monoacylglycerol and free fatty acids, crystallization and melting properties, emulsion stability and antimicrobial properties. The results confirm that the present enzyme-modified insect derived lipid compositions, surprisingly, combine increased dispersibility in water, a more favourable hardness profile (as a function of temperature) and a melting point slightly above ambient temperature. The compositions of the invention further have good nutritive properties as well as some interesting functional properties, e.g. anti-microbial effects. The products of the invention, accordingly, have a significantly improved potential for widespread application, compared to crude insect lipid compositions.

Hence, a first aspect of the present invention provides an enzyme-modified insect derived lipid composition comprising ≤95 wt. %, based on the total weight of the composition, of triacylglycerol (TAG); and/or ≥1 wt. %, based on the total weight of the composition, of diacylglycerol (DAG); and/or ≥2.5 wt. %, based on the total weight of the composition of free fatty acid (FFA).

A further aspect of the invention provides a method of producing an enzyme-modified insect lipid composition, said method comprising the steps of:
a) providing a crude insect lipid fraction;
b) adding to said crude insect lipid composition of step a), an enzyme capable of hydrolyzation and/or transesterification of TAG; and
c) keeping the composition as obtained in step b) under conditions favourable to enzyme activity.

A further aspect of the invention provides the enzyme-modified insect lipid composition obtainable by any one of the methods as disclosed herein.

Further aspects of the present invention provide (human or animal) alimentary products comprising an enzyme-modified insect lipid composition as disclosed herein.

Yet further aspects of the invention provide uses of the enzyme-modified insect lipid composition as disclosed herein for improving one or more properties of (human or animal) alimentary product.

These and other aspects of the invention, including exemplary and preferred embodiments thereof, will become apparent to those skilled in the art, based on the following description and examples.

### DETAILED DESCRIPTION OF THE INVENTION

A first aspect of the present invention provides the enzyme-modified insect lipid compositions per se.

As used herein, the term ‘insect lipid’ refers to any composition that is derived obtained and/or obtainable from insects and comprises primarily lipidic substances. The term “insects” refers to insects in any development stage, such as adult insects, insect larvae and insect pupae. A large variety of insects can be used. Preferably, edible insects or edible worms are used. More preferably, the insects are flies, bugs, mosquitos, butterflies, moths, cicadas, termites, bees, ants, wasps, beetles, grasshoppers, or crickets. More preferably, the insects belong to the species: black soldier fly (Hermetia illucens), house fly (Musca domestica), mealworm beetle (Tenebrio molitor), migratory locust (Locusta migratoria) and house cricket (Acheta domestica). In a particularly preferred embodiment of the invention, the insect lipid is a lipid derived/obtained from insects belonging to the species house fly, black soldier fly, morio worm, mealworm or cricket, and preferably black soldier fly (Hermetia illucens). In preferred embodiments, the insects are insect larvae, in particular larvae of the BSF.

The term ‘lipidic substance’ generally refers to any fatty, greasy, oily, or waxy substance that is not or only sparingly soluble in water but is soluble in non-polar solvents.

Insect derived lipid compositions comprise esters of fatty acids and glycerol as their main constituent. Hence, in accordance with preferred embodiments of the invention, the enzyme-modified insect lipid composition as defined herein primarily comprises fatty acid mono-, di- and tri-esters of glycerol and free fatty acids, e.g. in a combined amount of ≥ 50 wt. %, based on the total weight of the lipid composition. In accordance with preferred embodiments of the invention, an enzyme-modified insect lipid composition as defined herein is provided comprising fatty acid mono-, di- and tri-esters of glycerol and free fatty acids, in a combined amount of at least 60 wt. %, at least 65 wt. %, at least 70 wt. %, at least 75 wt. %, at least 80 wt. %, at least 85 wt. %, at least 90 wt. % or at least 95 wt. %, e.g. an amount within a range of 50-100 wt. %, 75-99.5 wt. %, or 85-99 wt. %, based on the total weight of the lipid composition. Exemplary other, non-lipidic components that may be present in the enzyme-modified insect lipid composition include glycerol, inactivated enzyme (residue), traces of moisture, etc.

As used herein, the term ‘enzyme-modified’ means modified by an enzyme and thus refers to any product obtainable by treating an insect derived lipid with an enzyme capable of modifying the lipidic components comprised therein, such as an esterase enzyme, a hydrolase enzyme, a lipase enzyme, an enzyme with transesterification activity, an enzyme with hydrolysis activity and combinations thereof. In preferred embodiments of the invention the enzyme has hydrolysis and (trans) esterification activity. In preferred embodiments of the invention the enzyme is non-regiospecific or weakly sn2 specific.

In accordance with preferred embodiments of the invention, an enzyme-modified insect lipid composition as defined herein is provided comprising≤95 wt. %, based on the total weight of the lipid composition, of triacylglycerol (TAG). As used herein, the term ‘triacylglycerol’ refers to esters composed of three fatty acids and glycerol, also often referred to as fatty acid tri-esters of glycerol and triglyceride, which terms are deemed synonymous and may be used interchangeably herein. As is apparent from the experimental section below, the amount of TAG in the enzyme-modified lipid is distinct from, notably lower than, the amount of TAG in the crude insect lipid. In accordance with preferred embodiments of the invention, an enzyme-modified insect lipid composition as defined herein is provided comprising TAG in an amount of less than 92.5 wt. %, less than 90 wt. %, less than 87.5 wt. % or less than 85 wt. % and/or in an amount of at least 25 wt. %, at least 40 wt. %, at least 50 wt. %, at least 55 wt. % or at least 60 wt. %, e.g. an amount within a range of 40-95 wt. %, 50-92.5 wt. %, or 55-90 wt. %, based on the total weight of the lipid composition.

In accordance with preferred embodiments of the invention, an enzyme-modified insect lipid composition as defined herein is provided comprising ≥1 wt. %, based on the total weight of the lipid composition, of diacylglycerol (DAG). As used herein, the term ‘diacylglycerol’ refers to esters composed of two fatty acids and glycerol, also often referred to as fatty acid di-esters of glycerol and diglyceride, which terms are deemed synonymous and may be used interchangeably herein. As is apparent from the experimental section below, the amount of DAG in the enzyme-modified lipid is distinct from, notably higher than, the amount of DAG in the crude insect lipid. In accordance with preferred embodiments of the invention, an enzyme-modified insect lipid composition as defined herein is provided comprising DAG in an amount of at least 1 wt. %, at least 2.5 wt. %, at least 4 wt. %, at least 5 wt. % or at least 6 wt. % and/or in an amount of less than 20 wt. %, less than 15 wt. %, less than 12.5 wt. % or less than 10 wt. %, e.g. an amount within a range of 4-15 wt. %, 5-12.5 wt. %, or 6-10 wt. %, based on the total weight of the lipid composition.

In accordance with preferred embodiments of the invention, an enzyme-modified insect lipid composition as defined herein is provided comprising ≥1 wt. %, based on the total weight of the lipid composition, of monoacylglycerol (MAG). As used herein, the term ‘diacylglycerol’ refers to esters composed of one fatty acids and glycerol, also often referred to as fatty acid mono-esters of glycerol and monoglyceride, which terms are deemed synonymous and may be used interchangeably herein. As is apparent from the experimental section below, the amount of MAG in the enzyme-modified lipid is distinct from, notably higher than, the amount of MAG in the crude insect lipid. In accordance with preferred embodiments of the invention, an enzyme-modified insect lipid composition as defined herein is provided comprising MAG in an amount of at least 0.05 wt. %, at least 0.1 wt. %, at least 0.25 wt. %, at least 0.4 wt. %, at least 0.5 wt. % or at least 0.6 wt. % and/or in an amount of less than 5 wt. %, less than 2.5 wt. %, less than 1 wt. % or less than 0.75 wt. %, e.g. an amount within a range of 0.05-5 wt. %, 0.1-2.5 wt. %, or 0.1-1 wt. %, based on the total weight of the lipid composition.

In accordance with preferred embodiments of the invention, an enzyme-modified insect lipid composition as defined herein is provided comprising ≥2.5 wt. %, based on the total weight of the lipid composition, of free fatty acids (FFA). As is apparent from the experimental section below, the amount of FFA in the enzyme-modified lipid is distinct from, notably higher than, the amount of FFA in the crude insect lipid. In accordance with preferred embodiments of the invention, an enzyme-modified insect lipid composition as defined herein is provided comprising FFA in an amount of at least 2.5 wt. %, at least 4 wt. %, at least 5 wt. %, at least 6 wt. %, at least 7 wt. %, at least 8 wt. %, at least 9 wt. % or at least 10 wt. %, and/or in an amount of less than 40 wt. %, less than 35 wt. %, less than 30 wt. % or less than 25 wt. %, e.g. an amount within a range of 2.5-40 wt. %, 5-35 wt. %, or 8-30 wt. %, based on the total weight of the lipid composition.

In accordance with preferred embodiments of the invention, an enzyme-modified insect lipid composition as defined herein is provided, comprising C12:0 fatty acid (lauric acid) in an amount of at least 35%, based on total fatty acid content, preferably at least 40%, at least 42.5%, or at least 45% and/or in an amount of less than 60%, less than 55%, less than 52.5% or less than 50%, e.g. an amount within the range of 40-60%, based on the total fatty acid content.

Whenever, in this document, the relative amount of a specific fatty acid is defined, it is given as the percentage based on weight (which may also be referred to as ‘% (w/w)’, ‘wt. %’, etc.). In defining the fatty acid composition of the products of the invention, no distinction is made between fatty acids esterified to glycerol (as MAG, DAG or TAG) and free fatty acids, unless indicated otherwise.

In accordance with preferred embodiments of the invention, an enzyme-modified insect lipid composition as defined herein is provided, comprising C16:0 fatty acid (palmitic acid) in an amount of at least 5%, based on total fatty acid content, preferably at least 6%, at least 7%, at least 7.5% or at least 8% and/or in an amount of less than 25%, less than 22.5%, less than 20% or less than 17.5%, e.g. an amount within the range of 5-22.5%, based on the total fatty acid content.

In accordance with certain embodiments of the invention, an enzyme-modified insect lipid composition as defined herein is provided, comprising C18:1 fatty acid (oleic acid) in an amount of at least 4%, based on total fatty acid content, preferably at least 5%, at least 6% or at least 7% and/or in an amount of less than 20%, less than 15%, less than 12.5% or less than 11%, e.g. an amount within the range of 4-20%, based on the total fatty acid content.

In accordance with certain embodiments of the invention, an enzyme-modified insect lipid composition as defined herein is provided, comprising C18:2 fatty acid (linoleic acid) in an amount of at least 5%, based on total fatty acid content, preferably at least 7%, at least 8% or at least 9% and/or in an amount of less than 25%, less than 20%, less than 18% or less than 16%, e.g. an amount within the range of 9-16%, based on the total fatty acid content.

In accordance with certain embodiments of the invention, an enzyme-modified insect lipid composition as defined herein is provided, comprising 15-40%, based on total fatty acid content, of unsaturated fatty acids. In accordance with preferred embodiments of the invention, an enzyme-modified insect lipid composition as defined herein is provided comprising unsaturated fatty acids in amount of at least 17.5%, at least 20%, at least 21% or at least 22% and/or in an amount of less than 35%, less than 32.5%, less than 30% or less than 28%, based on the total fatty acid content.

In accordance with certain embodiments of the invention, an enzyme-modified insect lipid composition as defined herein is provided, comprising 5-20%, based on total fatty acid content, of omega-9 fatty acids. In accordance with preferred embodiments of the invention, an enzyme-modified insect lipid composition as defined herein is provided comprising omega-9 fatty acids in amount of at least 6%, at least 7%, at least 7.5% or at least 8% and/or in an amount of less than 17.5%, less than 15%, less than 12.5% or less than 12%, based on the total fatty acid content.

In accordance with preferred embodiments of the invention, an enzyme-modified insect lipid composition as defined herein is provided, having a melting point of above 15° C., as determined by DSC analysis, e.g. using a DSC Q2000 (TA Instruments) with aluminum hermetic pans, wherein heat flow of the sample is measured during a 1st heating run, a cooling run and a 2nd heating run from −10° C. until 60° C. with 10° C./min. In preferred embodiments of the invention, the enzyme-modified insect lipid composition has a melting point of above 17.5° C., above 20° C., above 21° C., above 22° C., above 22.5° C., or above 23° C. as determined by DSC analysis. The melting point is typically below 35° C., e.g. below 30° C., below 27.5° C., below 26° C., below 25° C. or below 24° C.

As already mentioned herein, the enzyme-modified insect lipid composition of the invention has a very favourable hardness profile, as a function of temperature. More in particular, as illustrated in the experimental part, the hardness of the enzyme-modified insect lipid composition of the invention at temperatures within the range of 0-30° C. is significantly reduced compared to that of crude insect derived lipid, as established by a (hedonic) knife-penetration test. Without wishing to be bound by any particular theory, the inventors believe that this reduction in hardness at the given temperature can be correlated with a reduced solid fat content at the given temperatures determined using NMR spectroscopy, as described in the experimental part as well.

Hence in certain embodiments of the invention, enzyme-modified insect lipid compositions are provided having solid fat content at a temperature of 10° C., of less than 55 wt. %, such as less than 52.5 wt. %, less than 50 wt. %, less than 47.5 wt. %, less than 45 wt. % less than 42.5 wt. % or less than 40 wt. %. In certain embodiments, the solid fat content at a temperature of 10° C. is at least 15 wt. %, such as at least 20 wt. %, at least 25 wt. %, at least 27.5 wt. % or at least 30 wt. %.

In certain embodiments of the invention, enzyme-modified insect lipid compositions are provided having solid fat content at a temperature of 20° C., of less than 30 wt. %, such as less than 25 wt. %, less than 20 wt. %, less than 17.5 wt. %, less than 15 wt. % or less than 14 wt. %. In certain embodiments, the solid fat content at a temperature of 20° C. is at least 1 wt. %, such as at least 2.5 wt. %, at least 5 wt. %, at least 7.5 wt. %, at least 10 wt. %, at least 12.5 wt. % or at least 15 wt. %.

In certain embodiments of the invention, enzyme-modified insect lipid compositions are provided having solid fat content at a temperature of 25° C., of less than 14 wt. %, such as less than 10 wt. %, less than 5 wt. %, less than 2.5 wt. %, less than 2 wt. % or less than 1.75 wt. %. In certain embodiments, the solid fat content at a temperature of 25° C. is at least 0.1 wt. %, such as at least 0.5 wt. %, at least 0.75 wt. %, at least 1 wt. %, at least 1.25 wt. % or at least 1.5 wt. %

In accordance with preferred embodiments of the invention, an enzyme-modified insect lipid composition as defined herein is provided, that has improved dispersibility in water or an aqueous liquid. In one embodiment of the invention an enzyme-modified insect lipid composition as defined herein is provided, that has improved emulsion stability in water, compared to crude insect derived lipid, as reflected by a delay in the occurrence of phase separation. In certain embodiments of the invention, the enzyme-modified insect lipid compositions are characterized by the ability to form stable emulsions with demi water and a suitable surfactant, such as with a surfactant comprising Span 20 and polyoxyl 35 castor oil at a 1:1 weight ratio, with a lipid:water:surfactant ratio of 3:95:2. Typically, such emulsions based on the enzyme-modified insect lipid composition of the invention do not show phase separation, when kept at 20° C., for a period of at least 2 days, e.g. at least 3 days, at least 4 days or at least 5 days, whereas the corresponding emulsion based on crude insect derived lipid (visually) shows phase separation after about a day. In other embodiments of the invention the % phase separation, as calculated on the basis of the height of the separated fraction (in a measuring cylinder), after standing for 5 days at 20° C., is less than 5%, preferably less than 4%, less than 3%, less than 2.5%, less than 2%, less than 1.5%, less than 1%, less than 0.5% or less than 0.25%.

In another aspect, the present invention provides a method of producing an enzyme-modified insect lipid composition, said method comprising the steps of:
a) providing a crude insect lipid fraction;
b) adding to said crude insect lipid composition of step a), an enzyme capable of hydrolyzation and, optionally, transesterification of TAG; and
c) keeping the composition as obtained in step b) under conditions favorable to enzyme activity.

Methods of producing a crude lipid fraction from insects are known to those skilled in the art. Although the invention is not particularly limited in this regard, embodiments are provided, wherein step a) comprises:
a1) obtaining a pulp from insects;
a2) heating the pulp to a temperature of at least 70° C.; and
a3) subjecting the heated pulp to a physical separation step to produce the crude insect lipid fraction.

In the international patent application WO2014/123420, incorporated herein by reference, a general method has been described for obtaining nutrient streams, including a crude lipid fraction, from insects. In preferred embodiments of the present invention, step a) of the method, i.e. the provision of a crude insect lipid composition, is based on said method. Hence, in preferred embodiments of the invention, methods of producing an enzyme-modified insect lipid composition as defined herein are provided, wherein step a) comprises:
a1) obtaining a pulp from insects, wherein the insects or worms are reduced in size, e.g. to a particle size of less than 1 mm;
a2) heating the pulp to a temperature of at least 70° C., preferably 70-100° C., and
a3) subjecting the heated pulp to a physical separation step, such as centrifugation and/or decantation, thereby obtaining a crude lipid fraction, an aqueous protein fraction and a solid-containing fraction.

In the international patent application WO2021/125956, incorporated herein by reference, improved methods are disclosed to provide nutrient streams similar to those produced in accordance with WO2014/123420, but with a significantly higher output/yield. Hence, in preferred embodiments of the invention, methods of producing an enzyme-modified insect lipid composition as defined herein are provided, wherein step a) comprises:
a1) providing insects and preparing a pulp thereof;
a2) heating the insect pulp for 50-100 seconds at a temperature of 60° C.-95° C., therewith providing the nutrient stream consisting of heated insect pulp; and
a3): subjecting the heated insect pulp of step a2) to a physical separation step thereby obtaining a crude lipid fraction, an aqueous protein fraction and a solid-containing fraction.

In yet a further preferred embodiments of the invention, methods of producing an enzyme-modified insect lipid composition as defined herein are provided, wherein step a) comprises:
a1) obtaining a pulp from insects:
   providing insect larvae that have been fed a vegetal diet and/or a microbial diet;
   starving the insect larvae for at least 10 minutes;
   washing the starved insect larvae with an aqueous solution, preferably with water;
   reducing the insect larvae in size, therewith providing a slurry of minced insect larvae;
a2) heating the slurry obtained in step (a1) to a temperature of at least 70° C.;
a3) extracting a lipid fraction from the heated slurry of step (a2) by
   subjecting the heated slurry to a first separation step at a temperature of at least 70° C. so as to obtain a first lipid fraction;
   separating water and non-fat soluble solids from the first lipid fraction obtained in the previous step, by subjecting the first lipid fraction to a second separation step at a temperature of at least 55° C., so as to obtain the crude insect lipid composition.

In preferred embodiments of the invention, a method as defined herein is provided, wherein the vegetal diet of step a1) comprises wheat or potato or a mixture of wheat and potato, preferably in a weight ratio of between 20:1 and 1:20, such as in a weight ratio of between 15:1 and 1:15; between 10:1 and 1:10; between 6:1 and 1:6; between 4:1 and 1:4; between 3:1 and 1:3; or between 2:1 and 1:2, such as about 1:1. Furthermore, in preferred embodiments of the invention, a method as defined herein is provided, wherein
the insect larvae provided in step a1) are at least 5 days of age, preferably 8-15 days of age, more preferably 9-13 days of age, most preferably 10-12 days of age, for example 5, 6, 7, 8, 9, 10, 11, 12, 13, 14, 15, 16 or 17 days of age since hatching. Furthermore, in preferred embodiments of the invention, a method as defined herein is provided, wherein step a1) comprises starving the insect larvae at age 9-13 days, typically 10-12 days. In preferred embodiments, the starvation lasts for 10 minutes to 4 weeks, preferably for 30 minutes to 1 week, more preferably for 60 minutes to 120 hours, most preferably for between 3 hours and 1 day. In preferred embodiments of the invention step a1) comprises storing the insect larvae, following starvation, at 8° C.-15° C., preferably 10° C.-12° C., typically for a period of up to three weeks or up to one week, such as 1 day, 2 days, 3 days, 4 days or 5 days, before subjecting the starved and stored larvae to the subsequent step of the method. Furthermore, in preferred embodiments of the invention, a method as defined herein is provided, wherein the aqueous wash solution in step (a1) is water, preferably water having a temperature of between 10° C. and 30° C., more preferably between 15° C. and 27° C., most preferably between 18° C. and 23° C., such as about 20° C. Furthermore, in preferred embodiments of the invention, a method as defined herein is provided, wherein the insect larvae are reduced in size in step a1) by mincing and/or the insect larvae are reduced in size to pieces of 0.5 mm or smaller, such as pieces having an average particle size within the range of between 10 and 500 micron, preferably by cutting. Furthermore, in preferred embodiments of the invention, a method as defined herein is provided, wherein, in step a2), the temperature is at least 90° C., preferably between 90° C. and 105° C., more preferably between 95° C. and 100° C.; and/or In step (a2) of the method, the temperature during heating of the minced larvae slurry is for example about 92° C., about 93° C., about 96° C., about 97° C. or about 99° C. Furthermore, in preferred embodiments of the invention, a method as defined herein is provided, wherein, in step (a3), the first separation step is a first centrifugation step wherein the applied g-force is between 3.000*g and 4.000*g applied for at least 1 minute, preferably about 3.500*g, preferably applied for at least 2 minutes, such as between 1 minute and 30 minutes, and/or the temperature in step (a3) is between 78° C. and 95° C., preferably between 80° C. and 90° C. Furthermore, in preferred embodiments of the invention, a method as defined herein is provided, wherein, in step (a3), the second separation step is a second centrifugation step wherein the applied g-force is between 9.000*g and 13.000*g applied for at least 1 minute, preferably about 11.000*g, preferably applied for at least 2 minutes, such as between 1 minutes and 30 minutes, preferably about 2 minutes, and/or the temperature in step (g) is between 58° C. and 90° C., preferably between 60° C. and 80° C. Furthermore, in preferred embodiments of the invention, a method as defined herein is provided, wherein the insect pulp is not enzymatically treated prior to heating in step a2). In other embodiments, a method as defined herein is provided wherein step a1) further comprises a step of treating the insect pulp with an enzyme prior to step a2), typically a peptidase, preferably a mixture of at least one protease and at least one peptidase, such as Flavourzyme. Such enzyme treatment may comprise treating the insect pulp with the enzyme for a period of between 0.5 and 3 hours, preferably between 1 and 2 hours, at a temperature of between 40° C. and 70° C., preferably between 45° C. and 65° C., more preferably at a temperature of 50° C.±2° C.

The processes of providing crude insect lipid fractions as defined herein can be carried out in a continuous method, at a scale that provides sufficient amount to render the present methods economically viable. The methods set forth here above, comprise steps that altogether result in a very stable and robust way of providing lipids of constant quality, the lipids having constant properties from batch to batch or in a continuous method, from time to time, and at controllable rate. Yields are typically achievable with the current extraction methods of at least 40%, and typically at least 60% lipids, based on the total fat content of the larvae applied in the method, while insect larvae such as larvae of BSF, comprise a fat content of about 40%, so that the extraction method is suitable for provision of lipids at a scale that is economically feasible.

As already explained herein before, in accordance with the present invention, the crude insect lipid fraction as produced in step a) is treated with a lipid modifying enzyme, such as an enzyme with an enzyme with hydrolysis activity and, optionally, transesterification activity. Hence, in preferred embodiments of the invention, step b) comprises the addition of an enzyme selected from the group consisting esterase enzymes, hydrolase enzymes, lipase enzymes, an enzyme with transesterification activity, an enzyme with hydrolysis activity and combinations thereof. In some embodiments, the enzyme component can include (exogenous) triacylglycerol lipase, (exogenous) carboxylic ester hydrolase, and combinations thereof. In preferred embodiments of the invention the enzyme has hydrolysis and (trans) esterification activity. In preferred embodiments of the invention the enzyme is non-regiospecific or weakly sn2 specific. In preferred embodiments, the enzyme component includes (exogenous) triacylglycerol lipase activity and/or (exogenous) carboxylic ester hydrolase activity, in combination with (trans) esterification activity, preferably in a non-regiospecific or weakly sn2 specific manner. Enzyme preparations that can suitably be used in accordance with the present invention are commercially available, for example, from Novozymes under the tradename Eversa™ Transform 2.0 enzyme. Esterase enzyme can be added to the crude insect lipid composition as obtained in step a), in a range of concentration. The (relative)

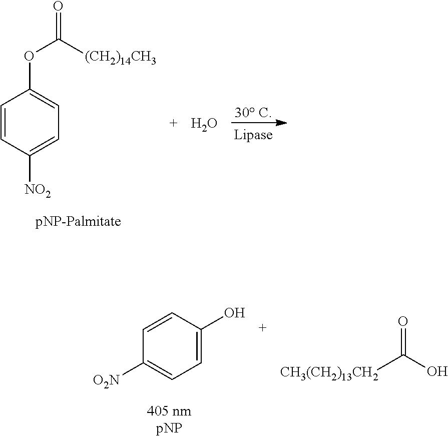

amount of esterase enzyme needed to produce a desired product can vary depending on factors such as one or more of the enzyme composition that is selected, the pH of the composition during the treatment, the temperature of the mixture during treatment, the time period of treatment, the amount of endogenous esterase enzyme present in the crude lipid composition, etc. In preferred embodiments of the invention, the crude insect lipid composition and enzyme component are combined at amounts or ratio's resulting in at least 50 LCLU of enzyme activity per 100 g of lipid composition, e.g. at least 500 LCLU/kg, at least 1000 LCLU/kg or at least 10000 LCLU/kg, such as 50-100000 LCLU/kg, 100-50000 LCLU/kg or 500-10000 LCLU/kg. In the context of the present invention, the lipolytic activity is defined as long chain lipase units (LCLU), using pNP-Palmitate (C: 16) as substrate. The method is based on the hydrolysis of pNP-palmitate by the enzyme, when incubated at pH 8.0, 30° C., to release pNP, according to the reaction scheme depicted below, which is yellow and can be detected at 405 nm.

According to the invention, one LCLU is the amount of enzyme which, under said conditions, liberates 1 micromol of pNP per minute.

In preferred embodiments of the invention, step c) comprises incubation of the mixture as obtained in step b) so as to allow the enzyme to at least partially hydrolyse fatty acid glycerol esters present in the crude insect lipid compositions. In embodiments of the invention, step c) comprises incubation of the mixture as obtained in step b) so as to allow the enzyme to convert the crude insect lipid composition into the enzyme-modified insect lipid composition having the specifications as defined herein before. In embodiments of the invention, step c) comprises incubation of the mixture as obtained in step b) at a temperature within the range of 20° C. to 50° C., such as a temperature within the range of 25° C. to 45° C., a temperature within the range of 30-40° C., a temperature within the range of 32.5-37.5° C., a temperature of 35° C.±2° C., a temperature of about 35° C. or a temperature of 35° C. In embodiments of the invention, step c) comprises incubation of the mixture as obtained in step b) at any such temperature for a period of 15 minutes to 5 hours, such as 30 minutes to 4 hours, 60-180 minutes, 90-150 minutes, or about 120 minutes. In a specific embodiment of the invention, step c) comprises incubation of the mixture as obtained in step b), having an enzyme concentration of 50-1000 LCLU-SL/100 g crude lipid composition, at a temperature of 30-40° C. for a period of 60-180 minutes, e.g. incubation of the mixture as obtained in step b), having an enzyme concentration of 100-1000 LCLU-SL/100 g crude lipid composition, at a temperature of 32.5-37.5° C. for a period of 90-150 minutes.

After the reaction mixture has been incubated for a period long enough to attain the extent of lipolysis desired or a period long enough to obtain the enzyme-modified insect lipid composition having the specifications as defined herein before, it is typically subjected to a treatment aimed at the destruction of the enzyme and/or the (permanent) inhibition of enzyme activity. Hence in preferred embodiments of the invention, a method as defined herein is provided, further comprising a step d), wherein the lipid composition as obtained in step c) is subjected to enzyme inhibition conditions, such as heating the composition to a temperature of at least 60° C., at least 70° C., at least 80° C. or at least 90° C., e.g. for a period of time of at least 1 minute, at least 2.5 minutes, at least 5 minutes, at least 7.5 minutes or at least 10 minutes.

As will be understood by those skilled in the art, based on the present teachings, the product as obtained after step c) or d) may be subjected to further processing steps, such as any further work-up step conventionally applied in the manufacturing of lipid compositions, e.g. degumming, fractionation deodorization, bleaching, packaging, etc.

Another aspect of the present invention concerns a product that is obtainable by any one of the methods defined herein. As will be understood by those skilled in the art, based on the present teachings, the inventors believe that the products that are obtainable by the present methods disclosed herein have the product specifications as defined here above (for the enzyme-modified insect lipid composition). The invention is not limited by any theory though, and the scope extents to any product that is obtainable by the methods disclosed herein, including products that prove to be outside some or all of product specifications defined herein.

Another aspect of the invention, concerns a (human or animal) alimentary product comprising the enzyme-modified insect derived lipid composition of the invention. As used herein the term “alimentary product” refers to any product intended and suitable for consumption by (oral) ingestion by a human or animal, typically for nutritional and nourishment purposes and includes products selected from the group of human food products, pet food products, animal feed, etc., which may be in solid, semi-solid or liquid form. In accordance with preferred embodiments of the invention, an alimentary product as defined herein is provided, comprising the enzyme-modified insect lipid composition of the invention, at a relative amount of at least 1 wt. %, based on the total weight of the alimentary product, e.g. in an amount of at least 10 wt. %, at least 25 wt. %, at least 40 wt. %, at least 50 wt. %, at least 60 wt. %, at least 70 wt. %, at least 80 wt. % or at least 90 wt. % and/or in an amount of less than 100 wt. %, less than 99.5 wt. %, less than 99 wt. %, less than 98 wt. %, less than 97.5 wt. %, less than 95 wt. %, or less than 90 wt. %.

In one embodiment of the invention, the alimentary product is fat ball, for feeding birds, comprising the enzyme-modified insect lipid composition of the invention, preferably at a relative amount as defined here above, in combination with at least one further component selected from the group consisting of seeds, cereals, nuts, animal and vegetable oils/fats, starches and antioxidants, which at least one further component typically is present in the form of discrete particles evenly divided throughout a matrix constituted by the enzyme-modified insect lipid composition of the invention.

In one embodiment of the invention, the alimentary product is an o/w emulsion, comprising water, the enzyme-modified insect lipid composition of the invention, preferably at a relative amount as defined here above, and a surfactant, preferably a non-ionic surfactant, more preferably a non-ionic surfactant selected from the group consisting of Tween 20, Tween 40, Tween 60, Tween 80, Tween 85, Span 20, Span 40, Span 60, Span 80, poloxamer 188, Polysorbate 80, Polysorbate 20, Vit E-TPGS (TPGS), TPGS-1000, polyethyleneglycol-40 hydrogenated castor oil, ethoxylated castor oil, polyoxyethylene castor oil, poly(ethylene oxide) hydrogen castor oil, poly(ethylene oxide) stearic acid ester, polyethylene glycol stearate and mixtures thereof, more preferably a non-ionic surfactant selected from the group of Span 20 and ethoxylated castor oil and mixtures thereof. In another embodiment of the invention, a pre-mix is provided comprising the insect lipid composition of the invention in combination with the surfactant, which may be mixed with water to produce an o/w emulsion.

Other aspects of the present invention relate to certain uses of the enzyme-modified insect lipid composition as defined herein.

As is illustrated in the examples, the enzyme-modified insect lipid compositions of the invention possess significant anti-microbiological activity against various bacteria (and viruses), presumably owing to the presence of substantial quantities of lauric acid (and derivatives), especially monolaurin, and might be useful as an antimicrobial agent in food and feed products and/or as an agent to protect against microbial infection and control the balance and distribution of bacteria in human or animal gut microbiota.

Hence, in one aspect of the invention, the use is provided of the present enzyme-modified insect lipid compositions as an antimicrobial agent; as a preservative agent; for preventing microbial contamination, for slowing, preventing and/or inhibiting microbial growth and/or proliferation; for killing microbial organisms; etc.; in an alimentary product, such as an alimentary product as defined herein. In preferred embodiments, said microbial organisms are bacteria, preferably bacteria selected from the species, Clostridium perfringens, Escherichia coli, Clostridium difficile, Campylobacter jejuni, Listeria monocytogenes, Vibrio cholera, Salmonella typhi, enterotoxigenic Escherichia coli, etc.

Yet another aspect of the invention concerns an enzyme-modified insect lipid composition as defined herein for use as a medicament in a human or animal in need thereof. In a preferred embodiment of the invention, an enzyme-modified insect lipid composition as defined herein is provided for use in a method of treating and/or preventing microbial infection, such as microbial infections of the gastrointestinal tract, in a human or animal in need thereof, preferably in an animal such as swine, poultry, bovine, sheep, goats, birds, etc. In preferred embodiments, said microbial organisms are bacteria, preferably bacteria selected from the species, Clostridium perfringens, Escherichia coli, Clostridium difficile, Campylobacter jejuni, Listeria monocytogenes, Vibrio cholera, Salmonella typhi, enterotoxigenic Escherichia coli, etc.

The invention further provides methods of treating and/or preventing microbial infection, such as microbial infections of the gastrointestinal tract, in a human or animal in need thereof, preferably in an animal such as swine, poultry, bovine, sheep, goats, birds, etc., said method comprising the administration, preferably the oral administration, of an enzyme-modified insect lipid composition as defined herein.

Yet another aspect of the invention concerns an alimentary product as defined herein for use as a medicament in a human or animal in need thereof. In a preferred embodiment of the invention, an alimentary product as defined herein is provided for use in a method of treating and/or preventing microbial infection, such as microbial infections of the gastrointestinal tract, in a human or animal in need thereof, preferably in an animal such as swine, poultry, bovine, sheep, goats, birds, etc. In preferred embodiments, said microbial organisms are bacteria, preferably bacteria selected from the species, Clostridium perfringens, Escherichia coli, Clostridium difficile, Campylobacter jejuni, Listeria monocytogenes, Vibrio cholera, Salmonella typhi, enterotoxigenic Escherichia coli, etc.

The invention further provides methods of treating and/or preventing microbial infection, such as microbial infections of the gastrointestinal tract, in a human or animal in need thereof, preferably in an animal such as swine, poultry, bovine, sheep, goats, birds, etc., said method comprising the administration, preferably the oral administration, of an alimentary product as defined herein.

Unless otherwise defined, all terms used in disclosing the invention, including technical and scientific terms, have the meaning as commonly understood by one of ordinary skill in the art to which this invention belongs.

“A”, “an”, and “the” as used herein refer to both singular and plural forms unless the context clearly dictates otherwise. By way of example, “a compartment” refers to one or more than one compartment.

“Comprise”, “comprising”, “comprises” and “comprised of” as used herein are synonymous with “include”, “including”, “includes” or “contain”, “containing”, “contains” and are inclusive or open-ended terms that specify the presence of what follows, e.g. a component, and do not exclude or preclude the presence of additional, non-recited components, features, element, members, steps, known in the art or disclosed therein.

Furthermore, the various embodiments and features of embodiments, although referred to as “preferred” or “e.g.” or “for example” or “in particular” are to be construed as exemplary manners in which the invention may be implemented rather than as limiting the scope of the invention.

The recitation of numerical ranges by endpoints includes all numbers and fractions subsumed within that range, as well as the recited endpoints. The skilled person will appreciate that the present invention can incorporate any number of the specific features described above. “About” as used herein referring to a measurable value such as a parameter, an amount, a temporal duration, and the like, is meant to encompass variations of +/−10% or less, more preferably +/−5% or less, even more preferably +/−1% or less of and from the specified value, in so far such variations are appropriate to perform in the disclosed invention. However, it is to be understood that the value to which the modifier “about” refers is itself also specifically disclosed.

Throughout this text, the use of terms in brackets, usually means that the term within brackets specifies a possible option or a possible meaning and should thus not be considered limiting.

Advantages of the invention will become apparent from the following examples, which are given below as mere illustrations, and are non-limitative.

### DESCRIPTION OF THE FIGURES

FIG. 1. shows the melting temperature of triglycerides, diglycerides an monoglycerides from different fatty acids (glycerides containing same fatty acids).

FIG. 2. DSC curves of the cooling and 2nd heating run of Lipid 0.5%

FIG. 3. DSC curves of the cooling and 2nd heating run of Lipid 1%

FIG. 4. DSC curves of the cooling and 2nd heating run of Lipid 10%.

FIG. 5. DSC curves of the cooling and 2nd heating run of Lipid X.

FIG. 6. % Separation of oil to water emulsion of different lipid samples after standing at 20° C. for 1 to 7 days.

### EXAMPLES

### Example 1: Modified Insect Fat: Characteristics and Applications

### Background

Black soldier fly larvae (BSF) lipids are now being considered as sustainable alternative of palm kernel oil. Protix' LipidX is currently marketed/used in livestock feed and pet food formulations. A bird feed company performed a trial wherein LipidX was used for making fat balls for feeding birds. These fat balls proved to become very hard in winter temperatures. A livestock feed company performed a trial wherein LipidX was feeded through drinking water of poultry. It turned out that LipidX had insufficient solubility in water even with added surfactants.

A program was set up to investigate whether LipidX could be modified to meet the desire for a softer product with higher water dispersibility, without detriment to other chemical, physical and nutritional properties.

It was hypothesized that enzymatic processing of BSF lipids leading to production of partial glycerides and free fatty acids, and also possibly the re-esterification of fatty acids back on glyceride backbone could open several new applications. During this study, BSF lipids were enzymatically hydrolyzed and the resulting fractions were analysed for:
TAG, DAG, MAG and FFA composition of all hydrolysed product
Crystallization and melting properties
Emulsion stability
Antimicrobial properties

### Materials and Methods

A preliminary study had been conducted to compare different enzymes, all obtained from Novozymes, Denmark, which resulted in the selection of Eversa Transform 2.0. This enzyme was selected for in depth analysis. The details about this enzyme are as follows:

| Name of | | Temperature | | |
|---|---|---|---|---|
| enzyme | pH | (° C.) | Mode of action | Source |
| Eversa | 7 | 35 | Non-regiospecific | He et al. |
| transform | | | or weak sn-2 specific | (2016) |
| 2.0 | | | Activity: | Novozyme |
| | | | Hydrolysis | |
| | | | Esterification | |

### Enzymatic Hydrolysis

A batch of Eversa Transform 2.0 (NS-F1036) was sourced from Novozymes. This enzyme was stored according to the guidelines provided by the supplier. LipidX was obtained from Protix' production facility (stored according to specification) and used within 7 days of obtaining. Before hydrolysis LipidX was melted by keeping them in a water bath at 35° C. for 30 min. Following this hydrolysis was carried using following conditions:

| | | Concentration of Eversa | | | | Hydrolysis |
|---|---|---|---|---|---|---|
| S. | Sample | Transform 2.0 | | Time | Temp | inhibition |
| No. | name | (% wt) | LCLU-SL/100 g | (min) | (° C.) | conditions |
| 1 | Hydrolyzed | 0.5 | 50 | 120 | 35 | 10 min/90° C. |
| | lipid 0.5% | | | | | |
| 2 | Hydrolyzed | 1 | 100 | 120 | 35 | 10 min/90° C. |
| | lipid 1% | | | | | |
| 3 | Hydrolyzed | 10 | 1000 | 120 | 35 | 10 min/90° C. |
| | lipid 10% | | | | | |

Immediately after the hydrolysis the samples were transferred to fridge (4° C.) and stored there until further analysis.

### Knife Penetration Test (Hedonic Score)

All the samples were brought to room temperature (20° C.) and left for six hours to solidify. Samples (at 0, 10 and 20° C.) were presented to five respondents (2 males and 3 males, 25 to 35 year old) and asked to penetrate table knife into the solidified fat. They were asked to score lipids on below hedonic score:
1—Very soft
2—Slightly soft
3—Hard
4—Very hard
5—Extremely hard

### Composition Analysis of LipidX and Resulting Hydrolysed Lipid Fractions

Composition analysis was done at Nutrilab B.V. As a part of compositional analysis TAG, DAG, MAG and FFA content of LipidX and three hydrolysed fractions was analysed.

### Solid Fat Content (as a Function of Temperature)

Tubes are filled with molten product for each test/temperature. All tubes are first brought to 0° C. (stand for 60 minutes). From here, the tubes are transferred to water baths of the temperature to be measured (stand for 30 minutes). Then the tubes are measured by means of the NMR spectrometer to detemine solid fat content.

### Differential Scanning Calorimetry (DSC) to Analyse Crystallization and Melting Profile

DSC analysis was performed at PTG lab on a DSC Q2000 (TA Instruments) using aluminum hermetic pans. The heat flow of the sample was measured during a 1st heating, a cooling and a 2nd heating run from −10° C. until 60° C. with 10 K/min. In between the heating and cooling runs the temperature was kept isothermal for 5 minutes.

### Antimicrobial Activity Analysis

The spores of Bacillus stearothermophilus var. Calidolactis were incubated with test samples (coconut oil, LipidX, Hydrolysed lipids 0.5%, Hydrolysed lipids 1.0%, Hydrolysed lipids 10.0%) in environmental conditions and media indicated by the supplier to facilitate activation of viable bacteria. If the test samples have antimicrobial activity, then pH change and resulting colour change in the kit will happen differently in comparison to control. This relative colour change in comparison to control was used to judge the qualitative antimicrobial activity of samples.

### Emulsion Stability Testing

Oil-in-water emulsions were produced using coconut oil, LipidX, Hydrolysed lipids 0.5%, Hydrolysed lipids 1.0%, Hydrolysed lipids 10.0% by mixing oil samples, demi water and surfactant (50% Span 20+50% exthoxylated Castor oil) in ratio of 3:95:2 at 40° C. on a magnetic stirrer. The emulsions were poured in 100 ml measuring cylinders and allowed to stand for 7 days at 20° C. The height of separated fraction was measured using a scale to calculate % separation after 1, 2, 3, 4, 5, 6 and 7 days.

### Results and Discussion

### Knife Penetrate Test

Below table indicates individual and average score of respondents on hedonic scale:

| | Hydro- | Hydro- | Hydro- | | |
|---|---|---|---|---|---|
| | lysed | lysed | lysed | | |
| 20° C. | lipids | lipids | lipids | | Coconut |
| Respondent | 0.5% | 1% | 10% | LipidX | oil |
| R1 | 2 | 2 | 2 | 4 | 4 |
| R2 | 2 | 2 | 1 | 3 | 3 |
| R3 | 2 | 2 | 1 | 3 | 3 |
| R4 | 2 | 2 | 1 | 3 | 4 |
| R5 | 2 | 1 | 1 | 3 | 3 |
| Average | 2 | 1.8 | 1.2 | 3.2 | 3.4 |

Out of all the tested samples Hydrolysed lipids 10% was the softest. Whereas coconut oil and LipidX were the hardest sample. It appears that there was only minor difference in hardness between Hydrolysed lipids 0.5% and 1%.

| | Hydro- | Hydro- | Hydro- | | |
|---|---|---|---|---|---|
| | lysed | lysed | lysed | | |
| 10° C. | lipids | lipids | lipids | | Coconut |
| Respondent | 0.5% | 1% | 10% | LipidX | oil |
| R1 | 3 | 3 | 2 | 4 | 4 |
| R2 | 2 | 3 | 2 | 4 | 4 |
| R3 | 3 | 2 | 2 | 4 | 4 |
| R4 | 3 | 2 | 2 | 4 | 4 |
| R5 | 3 | 2 | 2 | 3 | 4 |
| Average | 2.8 | 2.4 | 2 | 3.8 | 4 |

All the lipids got harder with temperature going down. However, general trend was similar to that observed at 20° C.

| | Hydro- | Hydro- | Hydro- | | |
|---|---|---|---|---|---|
| | lysed | lysed | lysed | | |
| 0° C. | lipids | lipids | lipids | | Coconut |
| Respondent | 0.5% | 1% | 10% | LipidX | oil |
| R1 | 3 | 3 | 3 | 5 | 5 |
| R2 | 3 | 3 | 3 | 4 | 4 |
| R3 | 3 | 3 | 2 | 5 | 5 |
| R4 | 4 | 2 | 2 | 4 | 5 |
| R5 | 3 | 3 | 3 | 5 | 4 |
| Average | 3.2 | 2.8 | 2.6 | 4.6 | 4.6 |

Again, general trend was similar. However, LipidX got extremely hard at 0° C.

### Solid Fat Content (as a Function of Temperature)

Analysis of the three enzyme-treated products revealed that, at each temperature, the solid fat content was below that of the LipidX product, with the following order (of solid fat content): hydrolysed lipids 10%<Hydrolysed lipids 1%<Hydrolysed lipids 0.5%<LipidX, as summarized in the table below.

Solid fat content (% w/w)

| | | Hydrolyzed | Hydrolyzed | Hydrolyzed |
|---|---|---|---|---|
| Temperature | LipidX | lipid 0.5% | lipid 1% | lipid 10% |
| 10° C. | 57.5 | 49.2 | 47.1 | 31.1 |
| 20° C. | 32.2 | 17.7 | 13.4 | <0.5 |
| 25° C. | 14.2 | 1.6 | <0.5 | <0.5 |
| 30° C. | <0.5 | <0.5 | <0.5 | <0.5 |
| 35° C. | <0.5 | <0.5 | <0.5 | <0.5 |
| 40° C. | <0.5 | <0.5 | <0.5 | <0.5 |

### Composition Analysis

Results obtain are summarized in the table below:

| Respondent | TAG | DAG | MAG | FFA |
|---|---|---|---|---|
| Hydrolysed | 87.8 | 5.5 | 0 | 8.9 |
| lipids 0.5% | | | | |
| Hydrolysed | 84.3 | 6.5 | 0.1 | 10.9 |
| lipids 1% | | | | |
| Hydrolysed | 67.1 | 9.3 | 0.6 | 29.4 |
| lipids 10% | | | | |
| LipidX | 96.8 | 0.7 | 0 | 1.9 |

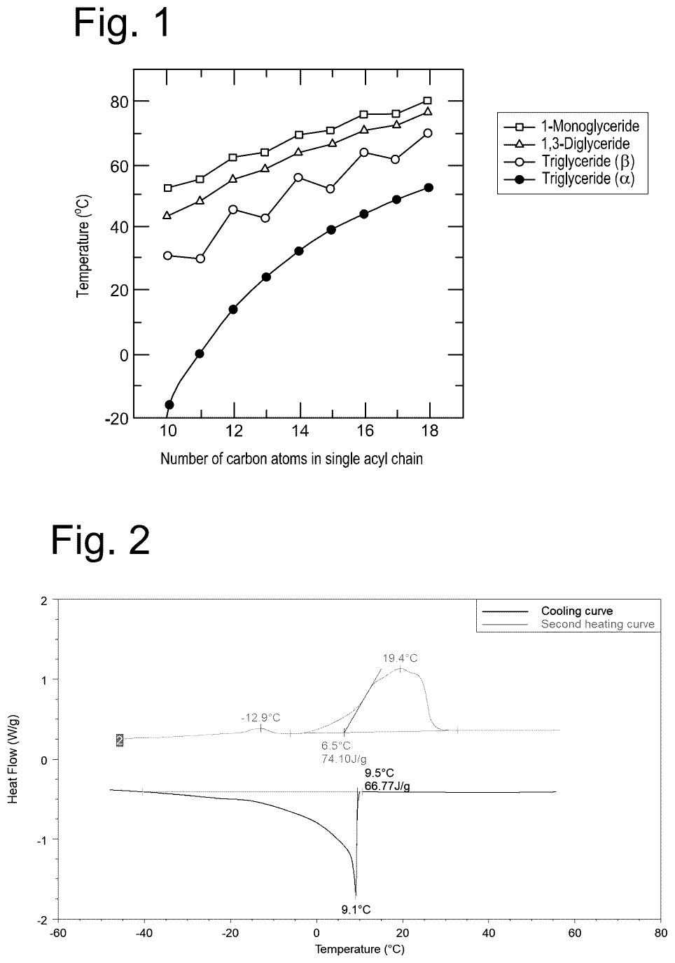

It is clearly visible that increasing the enzyme concentration results in an increase in the concentration of resulting diglycerides and free fatty acids. Another observation is that TAG, DAG, MAG and FFA content of Hydrolysed lipids 0.5% and 1% is very similar. Comparing these results with the results from the hardness testing, it can be seen that the sample with lowest TAG content is the softest and vice versa. According to Krog and Sparsø (2004, Food emulsifiers: their chemical and physical properties. Food Emuls. 45-91), monoglyceride and diglycerides have higher melting points than triglycerides. The melting point of lauric acid is 43.2° C. (PubChem, n.d.). Hence for lauric acid containing fatty acid derivatives, the order of melting temperature is: a crystals of trilaurin<β crystals of trilaurin<free lauric acid<1,3-dilaurin<1-monolaurin (see FIG. 1). It appears unlikely that reduction in hardness is due to molecules resulting from hydrolysis. Eversa transform 2.0 also possesses interesterification activity. It therefore appears that reduction in hardness of lipids after enzymatic processing is a result of interesterification (first hydrolysis of fatty acids and then esterification on glycerol backbone). It is hypothesized that the higher the production of partial glycerides and free fatty acids, the higher was the extent of interesterification. It is also hypothesized that production of high levels of DAG and MAG will improve the emulsifying properties of resulting fractions.

### DSC Profiles

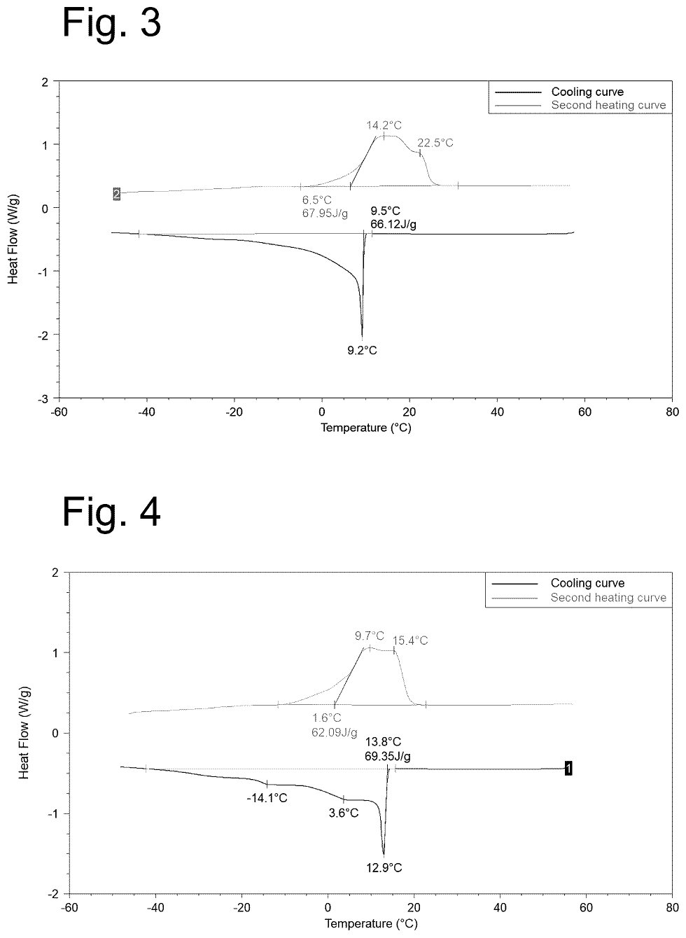

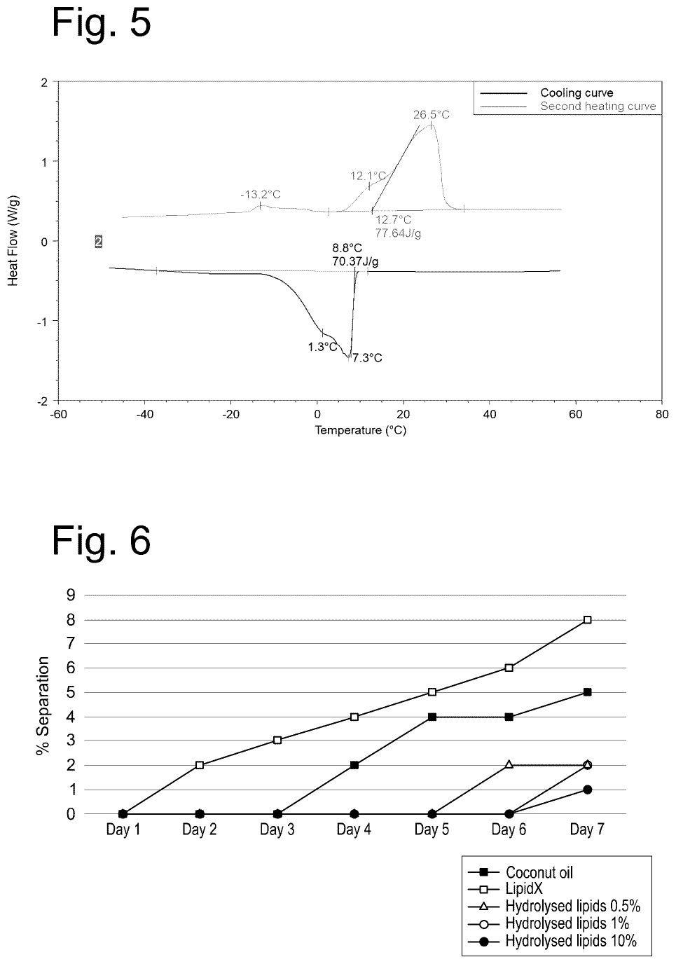

DSC profile of all the samples are shown in FIGS. 2-5. FIG. 2 shows the DSC cooling and 2nd heating curves of sample Lipid 0.5%. A crystallization peak is clearly visible at 9.1° C. The 2nd heating curve shows that the sample has a broad melting transition between, with a melting peak at 19.7° C. A smaller peak in the heating run occurs at −12.9° C. The DSC cooling and 2nd heating curves of sample Lipid 1% are shown in FIG. 3. A crystallization peak is clearly visible at 9.2° C. The 2nd heating curve shows that the sample has a broad melting transition with peaks at 14.2 and 22.5° C. The DSC cooling and 2nd heating curve of sample Lipid 10% are shown in FIG. 4. The cooling curve shows a sharp crystallization peak at 12.9° C. and some shoulder peaks at −14.1 and 3.6° C. The sample also has a broad melting transition with peaks at 9.7 and 15.4° C. The DSC cooling and 2nd heating curve of sample Lipid X are shown in FIG. 5. The cooling curve shows a crystallization transition with a peak at 7.3° C. and a shoulder peak at 1.3° C. The second heating curve shows a melting transition with peaks at 12.1 and 26.5° C. A small peak is also visible at −13.2° C. Highest peak of graph represents the temperature where largest proportion of fatty molecules melt. These results are consistent with the results indicated in previous sections, lower the TAG content, higher is the extent of interestification and lower is the temperature corresponding to the highest peak.

DSC integration limits of curves corresponding to different samples is indicated in below table:

| Sample | Heating run | Cooling run |
|---|---|---|
| Hydrolyzed lipids 0.5% | −6 to 32.7° C. | −40.7 to 10.7° C. |
| Hydrolyzed lipids 1% | −4.9 to 31° C. | −41.8 to 11.5° C. |
| Hydrolyzed lipids 10% | −11.6 to 22.8° C. | −41.8 to 15.8° C. |
| LipidX | −2.7 to 34.1° C. | −37.2 to 11.7 |

Here again it can be seen that hydrolyzed lipids 0.5% and 1% samples have very identical thermal profile.

### Antimicrobial Activity

The results of qualitative antimicrobial analysis of samples are mentioned in below table.

| Sample no. | Sample | Antimicrobial activity |
|---|---|---|
| 1 | Hydrolyzed lipids 0.5% | Detected |
| 2 | Hydrolyzed lipids 1% | Detected |
| 3 | Hydrolyzed lipids 10% | Detected |
| 4 | LipidX | Not detected |
| 5 | Coconut oil | Not detected |

It is clearly visible that triglyceride-based products i.e. LipidX and coconut oil have no antimicrobial activity in the above assay. Whereas all the hydrolyzed samples that containing free fatty acids and partial glycerides (including monoglycerides) have an antimicrobial activity. According to literature, monolaurin has the highest antimicrobial activity amongst all lauric acid derivatives. Free lauric acid also has considerable antimicrobial activity when compared to trilaurin (See Venugopal, V., 1999. PRESERVATIVES|Traditional Preservatives—Vegetable Oils, in: Robinson, R. K. (Ed.), Encyclopedia of Food Microbiology. Elsevier, Oxford, pp. 1743-1749). These results provide an indication that hydrolyzed insect fat used in this study could also be used to extend the shelf life of feed by exerting antimicrobial activity.

### Emulsion Stability Testing

The % separation data of emulsions made using tested oil samples after 1 to 7 days is indicated in FIG. 20.

It is clearly visible that LipidX formed the most unstable emulsion. Separation already started from day 2. After 7 days LipidX based emulsion showed 8% separation. Coconut oil emulsion started separating on day 3 and after 7 days 5% separation was observed.

Hydrolysed lipid-based emulsion were more stable than LipidX and coconut oil. Separation in Hydrolysed lipids 0.5, 1 and 10% started on day 5, 7 and 7, respectively. At the end of day 7, Hydrolysed lipids 0.5, 1 and 10% showed 2, 2 and 1% separation, respectively.

As a next step, further experiments were conducted using reduced surfactant concentrations of 1.5 and 1% to make emulsion. However, LipidX and Coconut oil did not give a stable emulsion at these concentrations. All three hydrolysed lipid samples produced a stable emulsion at 1.5 and 1% surfactant concentration respectively. The stability of these hydrolysed lipid emulsion were not measured (absent any control or benchmark).

This improved emulsifying ability of hydrolysed lipids could be attributed to the presence of higher levels of mono and di-glycerides in comparison to LipidX.

### CONCLUSIONS

During these studies, it was observed that hydrolysis of lipids resulted in increased concentration of partial glycerides and free fatty acids.

It was observed that hydrolysis treatment decreased the hardness of resulting fats at 0, 10 and 20° C. These findings were also in line with the results obtained using DSC analysis, where it was found that the hydrolyzed lipid samples started melting at a lower temperature. The enzymes used for hydrolysis possibly resulted in transesterification, which resulted in modification of melting and crystallization properties, These results are of significant interest for several applications. For example, production of fat balls for bird feeding, where balls are expected to stay solid, but still maintain a certain level of softness in winters. Another application could be production of cereal or energy bars for human consumption.

It was found that only hydrolyzed lipids have antimicrobial activity, which could be due to production of monoglycerides and free fatty acids (lauric derivatives). Indicating that hydrolyzed fats could be used to improve the microbial quality of animal feeds.

Additionally, it was observed that hydrolyzed lipid samples resulted in emulsion with better stability. This indicates that hydrolyzed lipids could be used for production of emulsified products, etc.

1. Enzyme-modified insect lipid composition comprising:
25-95 wt. %, based on the total weight of the composition, of triacylglycerol (TAG);
≥1 wt. %, based on the total weight of the composition, of diacylglycerol (DAG); and
2.5-35 wt. %, based on the total weight of the composition of free fatty acid (FFA).

2. Enzyme-modified insect lipid composition according to claim 1 comprising >0.1 wt. %, based on the total weight of the composition, of monoacylglycerol (MAG).

3. Enzyme-modified insect lipid composition according to claim 1 comprising 2.5-15 wt. %, based on the total weight of the composition, of DAG.

4. Enzyme-modified insect lipid composition according to claim 1, comprising 50-90 wt. %, based on the total weight of the composition, of TAG.

5. Enzyme-modified insect lipid composition according to claim 1, comprising 5-30 wt. %, based on the total weight of the composition, of FFA.

6. Enzyme-modified insect lipid composition according to claim 1, comprising between 40% and 60%, based on the total fatty acid content, of C12:0 fatty acid (lauric acid).

7. Enzyme-modified insect lipid composition according to claim 1, having a melting point within the range of 20-25° C.

8. Enzyme-modified insect lipid composition according to claim 1, having solid fat content at a temperature of 10° C., of less than 55 wt. % and/or solid fat content at a temperature of 20° C., of less than 25 wt. %.

9. Enzyme-modified insect lipid composition according to claim 1, which is an enzyme-modified lipid composition derived from black soldier flies (Hermetia illucens).

10. Method of producing an enzyme-modified insect lipid composition, said method comprising the steps of:
a) providing a crude insect lipid fraction;
b) adding to said crude insect lipid composition of step a), an enzyme capable of hydrolyzation and, optionally, transesterification of TAG; and
c) keeping the composition as obtained in step b) under conditions favorable to enzyme activity.

11. Method according to claim 10, wherein the crude insect lipid fraction is obtained by a process comprising the steps of:
a1) obtaining a pulp from insects;
a2) heating the pulp to a temperature of at least 70° C.; and
a3) subjecting the heated pulp to a physical separation step to produce a crude insect lipid fraction, therewith providing the crude insect lipid fraction of method step a).

12. Method according to claim 10, wherein the insect is black soldier fly, preferably black soldier fly larvae.

13. Method according to claim 10, wherein the enzyme the enzyme has hydrolysis and (trans) esterification activity.

14. Method according to claim 10, wherein step c) comprises incubation of the mixture as obtained in step b), having an enzyme concentration of 100-1000 LCLU-SL/100 g crude lipid composition, at a temperature of 30-40° C. for a period of 60-180 minutes.

15. (canceled)

16. (canceled)

17. (canceled)

18. (canceled)

19. (canceled)

20. (canceled)

21. (canceled)

22. Method of treating and/or preventing microbial infection, such as microbial infections of the gastrointestinal tract, in a human or animal in need thereof, comprising the administration to said human or animal of an enzyme-modified insect lipid composition comprising:
25-95 wt. %, based on the total weight of the composition, of triacylglycerol (TAG);
≥1 wt. %, based on the total weight of the composition, of diacylglycerol (DAG); and
2.5-35 wt. %, based on the total weight of the composition of free fatty acid (FFA); or
an alimentary product comprising the enzyme-modified insect lipid composition.

23. Method according to claim 22, wherein said human or animal is an animal selected from the group consisting of swine, poultry, bovine, sheep, goats and birds.

24. Method according to claim 22, wherein the microbial infection is an infection with bacteria selected from Clostridium perfringens, Escherichia coli, Clostridium difficile, Campylobacter jejuni, Listeria monocytogenes, Vibrio cholera, Salmonella typhi, and enterotoxigenic Escherichia coli.
